(19) United States
(12) Patent Application Publication
(10) Pub. No.: US 2022/0133346 A1
(43) Pub. Date: May 5, 2022

(54) ATHERECTOMY SYSTEM

(71) Applicant: BOSTON SCIENTIFIC SCIMED, INC., MAPLE GROVE, MN (US)

(72) Inventors: NICHOLAS ANASTASI, MINNEAPOLIS, MN (US); GARY THOMAS OHRT, CORCORAN, MN (US); MICHAEL KALAND, MINNEAPOLIS, MN (US)

(73) Assignee: BOSTON SCIENTIFIC SCIMED, INC., MAPLE GROVE, MN (US)

(21) Appl. No.: 17/513,959

(22) Filed: Oct. 29, 2021

Related U.S. Application Data
(60) Provisional application No. 63/107,996, filed on Oct. 30, 2020.

(51) Int. Cl. A61B 17/3207

(52) U.S. Cl. CPC A61B 17/320758

(57) ABSTRACT

Medical device systems as well as methods for making and using medical device systems are disclosed. An example medical device system may include an advancer including a drive mechanism. A sleeve may be secured to the drive mechanism. A drive shaft may extend through the sleeve. The drive shaft may have a proximal end region secured to the drive mechanism and a distal end region. A rotational device may be coupled to the distal end region of the drive shaft.

### CROSS REFERENCE TO RELATED APPLICATIONS

The present application claims the benefit of and priority to U.S. Prov. Pat. App. No. 63/107,996, filed Oct. 30, 2020 and titled ATHERECTOMY SYSTEM, the disclosure of which is incorporated herein by reference.

### TECHNICAL FIELD

The present disclosure pertains to medical devices, and methods for manufacturing and using medical devices. More particularly, the present disclosure pertains to rotational medical devices, methods, and systems.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A medical device system is disclosed. The medical device system comprises: an advancer including a drive mechanism; a sleeve secured to the drive mechanism; a drive shaft extending through the sleeve, the drive shaft having a proximal end region secured to the drive mechanism and a distal end region; and a rotational device coupled to the distal end region of the drive shaft.

Alternatively or additionally to any of the embodiments above, a distal section of the drive shaft extends distally from a distal end of the sleeve.

Alternatively or additionally to any of the embodiments above, the distal section has a substantially constant length.

Alternatively or additionally to any of the embodiments above, the advancer includes a housing and wherein the drive mechanism is configured to axially shift relative to the housing.

Alternatively or additionally to any of the embodiments above, the drive mechanism includes a control member for axially shifting the drive mechanism.

Alternatively or additionally to any of the embodiments above, the drive mechanism is configured to axially shift the drive shaft.

Alternatively or additionally to any of the embodiments above, the drive mechanism is configured to axially shift the sleeve.

Alternatively or additionally to any of the embodiments above, the drive shaft is axially fixed relative to the sleeve.

Alternatively or additionally to any of the embodiments above, the drive shaft includes a coil portion.

Alternatively or additionally to any of the embodiments above, the drive shaft includes a proximal portion coupled to the coil portion.

A rotational atherectomy system is disclosed. The rotational atherectomy system comprises: a catheter including a sleeve; a coiled drive shaft extending through the sleeve, the coiled drive shaft having a proximal end region and a distal end region; a rotational atherectomy device coupled to the distal end region of the coiled drive shaft; a proximal drive shaft coupled to the proximal end region of the coiled drive shaft; a drive mechanism coupled to the catheter; wherein the proximal drive shaft is attached to the drive mechanism; and wherein the sleeve is attached to the drive mechanism.

Alternatively or additionally to any of the embodiments above, a fixed-length section of the coiled drive shaft extends distally from a distal end of the sleeve.

Alternatively or additionally to any of the embodiments above, the coiled drive shaft is axially fixed relative to the sleeve.

Alternatively or additionally to any of the embodiments above, the proximal drive shaft is axially fixed relative to the sleeve.

Alternatively or additionally to any of the embodiments above, the drive mechanism includes a control member for axially shifting the coiled drive shaft.

A rotational atherectomy system is disclosed. The rotational atherectomy system comprises: a drive assembly including a drive mechanism and an actuator coupled to the drive mechanism; a catheter coupled to the drive mechanism, the catheter including a sleeve; a coiled drive shaft extending through the sleeve; an atherectomy burr coupled to a distal end region of the coiled drive shaft; wherein the sleeve is axially-fixed relative to the coiled drive shaft; and wherein a fixed-length section of the coiled drive shaft extends distally from a distal end of the sleeve.

Alternatively or additionally to any of the embodiments above, further comprising a proximal drive shaft coupled to a proximal end region of the coiled drive shaft.

Alternatively or additionally to any of the embodiments above, the proximal drive shaft is attached to the drive mechanism.

Alternatively or additionally to any of the embodiments above, the coiled drive shaft is axially fixed relative to the sleeve.

Alternatively or additionally to any of the embodiments above, the sleeve is attached to the drive mechanism.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a plan overview of an example medical device system.

FIG. 2 is a partially cutaway view of a portion of an example medical device system.

FIG. 3 is a partially cutaway view of a portion of an example medical device system.

FIG. 4 is a partially cutaway view of a portion of an example medical device system.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Cardiovascular disease and peripheral arterial disease may arise from accumulation of atheromatous material on the inner walls of vascular lumens, resulting in a condition known as atherosclerosis. Atheromatous and other vascular deposits may restrict blood flow and can cause ischemia in a heart of a patient, vasculature of a patient's legs, a patient's carotid artery, etc. Such ischemia may lead to pain, swelling, wounds that will not heal, amputation, stroke, myocardial infarction, and/or other conditions.

Atheromatous deposits may have widely varying properties, with some deposits being relatively soft and others being fibrous and/or calcified. In the latter case, the deposits may be referred to as plaque. Atherosclerosis occurs naturally as a result of aging, but may also be aggravated by factors such as diet, hypertension, heredity, vascular injury, and the like. Atherosclerosis may be treated in a variety of ways, including drugs, bypass surgery, and/or a variety of catheter-based approaches that may rely on intravascular widening or removal of the atheromatous or other material occluding the blood vessel. Atherectomy is a catheter-based intervention that may be used to treat atherosclerosis.

Atherectomy is an interventional medical procedure performed to restore a flow of blood through a portion of a patient's vasculature that has been blocked by plaque or other material. In an atherectomy procedure, a device on an end of a drive shaft is used to engage and/or remove (e.g., abrade, grind, cut, shave, etc.) plaque or other material from a patient's vessel (e.g., artery or vein). In some cases, the device on an end of the drive shaft may be abrasive and/or may otherwise be configured to remove plaque from a vessel wall or other obstruction in a vessel when the device is rotating and engages the plaque or other obstruction.

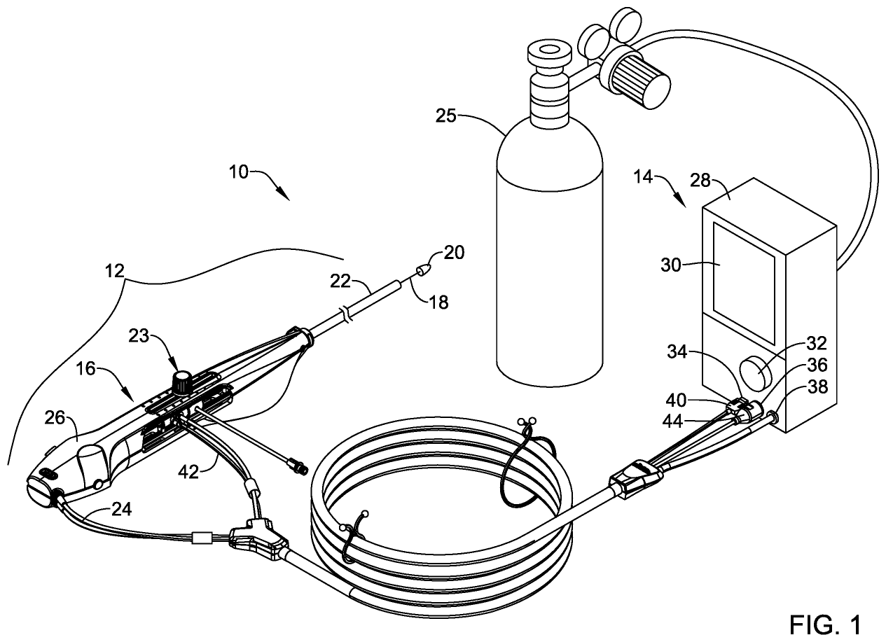

FIG. 1 depicts an example medical device system 10 (e.g., a rotational atherectomy system 10). The atherectomy system 10 may include a drive assembly 12 (e.g., an atherectomy drive assembly) and a control unit 14 (e.g., a controller or control console). Although the drive assembly 12 and the control unit 14 are depicted in FIG. 1 as separate components of the atherectomy system 10, the features of the control unit 14 may be incorporated into the drive assembly 12.

The drive assembly 12 may include, among other elements, a handle or advancer 16, a drive shaft 18 (e.g., a flexible drive shaft or other drive shaft), a rotational device 20 (e.g., a rotational burr or other rotational device), and a catheter or sleeve 22 having a first end (e.g., a proximal end), a second end (e.g., a distal end), and a lumen extending from the first end to the second end for receiving the drive shaft 18. The rotational device 20 may have a rough or sharp surface, such that it is configured to grind, abrade, cut, shave, etc. plaque from a vessel wall or other obstruction in a vessel when it is rotated.

The advancer 16 may include a drive mechanism 23 (e.g., a turbine, an electric motor, pneumatic motor, and/or one or more other suitable drive mechanisms) that is configured to move relative to (e.g., within, along, etc.) the advancer 16. The drive mechanism 23 may be configured to translate along a longitudinal path to longitudinally advance and/or retract the drive shaft 18 and/or and the rotational device 20 (e.g. relative to the advancer 16), secure the drive mechanism at an axial location along the longitudinal path, and/or adjust a mode of the drive mechanism.

The drive mechanism 23 may be coupled to the drive shaft 18 in a suitable manner including, but not limited to a weld connection, a clamping connection, an adhesive connection, a threaded connection, and/or other suitable connection configured to withstand high rotational speeds and forces. As the drive shaft 18 may rotate over a wide range of speeds (e.g., at speeds of between zero (0) rotations per minute (RPM) and 250,000 RPM or higher in a clockwise and/or counterclockwise direction), the coupling between the drive mechanism and the drive shaft 18 may be configured to withstand such rotational speed and associated forces.

In some cases, the drive mechanism may be in communication with the control unit 14. When in communication with the control unit 14, the drive mechanism may be in direct communication with the control unit (e.g., directly connected via wiring) or indirect communication (e.g., indirectly connected via multiple wiring connections and/or one or more devices). One example of indirect communication between a drive mechanism and the control unit 14 may include a drive mechanism (e.g., a turbine or pneumatic motor) powered by compressed air, where the control unit 14 may activate a compressed fluid flow from a cylinder 25 or other component to the drive mechanism (e.g., activate a valve of the control unit 14 or otherwise activate the compressed fluid flow), which may result in rotation of the drive mechanism and the drive shaft 18.

The drive shaft 18 may be formed from one or more of a variety of materials. For example, the drive shaft 18 may be formed from one or more of a variety of materials including steel, stainless steel, and/or other suitable materials.

The drive shaft 18 may have a suitable diameter and/or length for traversing vasculature of a patient. In some cases, the drive shaft 18 may have a diameter in a range from about 0.030 centimeters (cm) or smaller to about 0.150 cm or larger and a working length in a range from about ten (10) cm or shorter to about three hundred (300) cm or longer. Alternatively, the drive shaft 18 may have a different suitable diameter and/or a different suitable length.

The rotational device 20 may have an outer perimeter which is equal to or larger than a distal diameter of the drive shaft 18 and the sleeve 22. The rotational device 20 may have a symmetric design so that it penetrates equally well in both rotational directions, but this is not required and the rotational device 20 may be configured to penetrate in only one direction. The diameter of the drive shaft 18 may depend on the dimension of the lumen of the sleeve 22 and/or one or more other factors.

The rotational device 20 may be coupled to the drive shaft 18. Where the drive shaft 18 has a first end portion (e.g., a proximal end portion) and a second end portion (e.g., a distal end portion), the rotational device 20 may be coupled to the drive shaft 18 at or near the second end portion. In some cases, the rotational device 20 may be located at or adjacent a terminal end of the second end portion of the drive shaft 18.

The rotational device 20 may be coupled to the drive shaft 18 in any manner. For example, the rotational device 20 may be coupled to the drive shaft 18 with an adhesive connection, a threaded connection, a weld connection, a clamping connection, and/or other suitable connection configured to withstand high rotational speeds and forces. Similar to as discussed above with respect to the connection between the drive shaft 18 and the drive mechanism, as the drive shaft 18 and/or the rotational device 20 may rotate at speeds between zero (0) RPM and 250,000 RPM or higher in a clockwise direction, a counter clockwise direction, or both a clockwise direction and a counter clockwise direction, the coupling between the drive shaft 18 and the rotational device 20 may be configured to withstand such rotational speeds and associated forces.

The drive assembly 12 and the control unit 14 may be in communication and may be located in or may have a same handle/housing and/or located in or have separate housings (e.g., an advancer housing 26 and a control unit housing 28, respectively, or other housings). Whether in the same housing or in separate housings, the drive assembly 12 and the control unit 14 may be in communication through a wired (e.g., via one or more electrical lines 24) and/or a wireless connection. Wired connections may be made via one or more communication protocols including, but not limited to, USB, Ethernet, SPI, UART, HDMI, and/or any other suitable common or proprietary wired protocol, as desired. Wireless connections may be made via one or more communication protocols including, but not limited to, cellular communication, ZigBee, Bluetooth, WiFi, IrDA, dedicated short range communication (DSRC), EnOcean, and/or any other suitable common or proprietary wireless protocol, as desired.

Although not necessarily shown in FIG. 1, the drive assembly 12 may include and/or enclose one or more operational features in addition to those discussed above and/or as alternatives to those discussed above. For example, among other features, the drive assembly 12 may include control buttons, rubber feet, control electronics, drive circuitry, etc.

The control unit 14, which may be separate from the drive assembly 12 (e.g., as shown in FIG. 1) or may be included in the drive assembly 12, may include several features. For example, as shown in FIG. 1, the control unit 14 may include a display 30 and a control knob 32 (e.g., a drive mechanism speed (e.g., RPM or other speed) adjustment knob or other control knob). Additionally or alternatively, the control unit 14 may include one or more other features for controlling the drive mechanism and/or other features of the drive assembly 12 (e.g., one or more drive mechanism states) including, but not limited to, a processor, memory, input/output devices, a speaker, volume control buttons, on/off power supply switch, drive mechanism mode activation switch, a timer, a clock, and/or one or more other features.

The display 30 may be or may include any suitable type of display panel using any suitable display panel technology. For example, the display 30 may include one or more of the following types of display panels: Eidophor, Electroluminescent display (ELD), Electronic paper (E Ink, Gyricon), Light emitting diode display (LED), Cathode ray tube (CRT) (Monoscope), Liquid-crystal display (LCD) (TFT, LED, Blue Phase, IPS), Plasma display panel (PDP) (ALiS), Digital Light Processing (DLP), Liquid crystal on silicon (LCoS), Organic light-emitting diode (OLED) (AMOLED), Organic light-emitting transistor (OLET), Surface-conduction electron-emitter display (SED), Field emission display (FED), Laser TV (Quantum dot, Liquid crystal), MEMS display (IMoD, TMOS, DMS), Quantum dot display (QD-LED), Ferro liquid display (FLD), Thick-film dielectric electroluminescent technology (TDEL), Telescopic pixel display (TPD), Laser Phosphor Display (LPD), or other type of display panel. The display 30 may include a touch sensitive screen for receiving input, but this is not required.

The control knob 32 may be any suitable type of control knob. As depicted in FIG. 1, the control knob 32 may be a physical control knob that is adjusted (e.g., rotated or otherwise translated) to adjust a control feature (e.g., speed of rotation of the drive mechanism or other control feature). Alternatively or in addition, the control knob 32 may be physical buttons, a virtual control knob that may be adjusted by interacting with a touch sensitive surface, and/or other suitable component configured to be adjusted to adjust a control feature.

As depicted in FIG. 1, the control unit 14 may include one or more ports including, but not limited to, a fiber optic port 34, an electrical port 36, a fluid port 38, and/or one or more other ports. The fiber optic port 34 may be configured to receive a fiber optic connector 40 of a fiber optic line 42, where the fiber optic line 42 may be connected to and/or may be part of a position sensor configured to optically sense a position of the drive mechanism. Additionally or alternatively, other types of position sensors (e.g., tachometers) may be utilized that have different types of connections to the control unit 14. The electrical port 36 may be configured to receive an electrical connector 44 of the electrical line 24, where the electrical line 24 may be connected to and/or may be part of control electronics at the drive assembly 12. In some cases, the electrical line 24 may be directly connected to a main PCB of the drive assembly 12 and may be utilized to power an electrical assembly of the drive assembly 12. The fluid port 38 may be configured to receive a fluid line connector 46 of a fluid line 48, where the fluid line 48 may be in communication with the drive mechanism to power the drive mechanism. In instances when the drive mechanism is an electrical motor or a non-pneumatic drive mechanism, the fluid port 38, the fluid line connector 46, and/or the fluid line 48 may be omitted, but this is not required.

It can be appreciated that if the drive shaft 18 (e.g., and the rotational device 20 coupled thereto) moves relative to the sleeve 22, the length or distance that the rotational device 20 can be extended or “thrown” may be limited. In other words, in order to engage/treat a longer lesion, the advancer 16 may need to be shifted. In addition, the drive shaft 18 and/or the rotational device 20 may contact/interact with the sleeve 22 when attempting to shift their relative positions. The systems disclosed herein are designed so that the drive shaft 18 and the sleeve 22 are both coupled to the drive mechanism 23. This may help to increase the throw length of the device. Some of these and other features are disclosed herein.

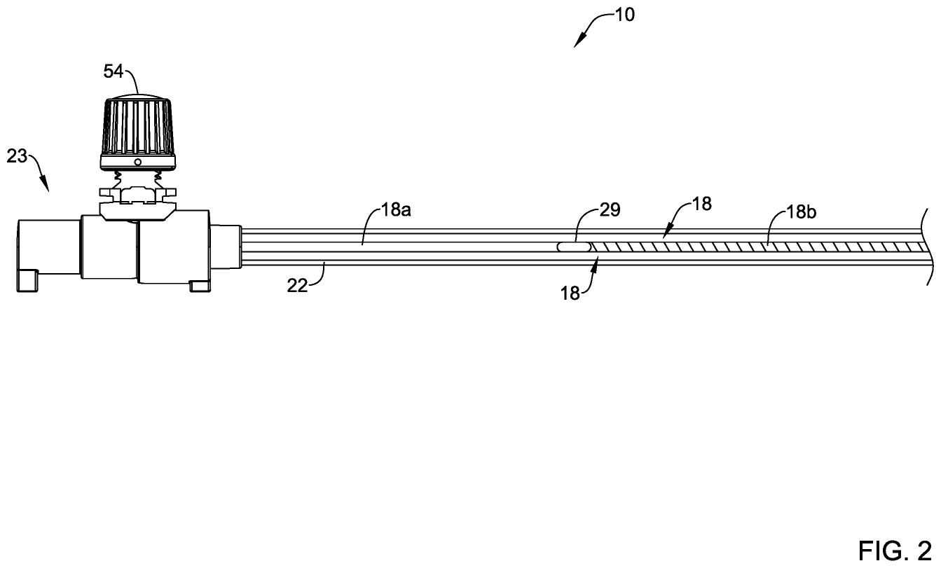

FIG. 2 is a partially cutaway view of a portion of the atherectomy system 10. Here the coupling/attachment of the drive shaft 18 and the sleeve 22 with/to the drive mechanism 23 is depicted. More particularly, the proximal end of the sleeve 22 and the proximal end of the drive shaft 18 are both secured to the drive mechanism 23. Because of this, actuation/translation of the drive mechanism 23 may move (e.g., translate) both the drive shaft 18 and the sleeve 22 together. Thus, a user may actuate an actuator or actuator portion 54 of the drive mechanism 23 to shift/translate the drive shaft 18 and the sleeve 22. It can be appreciated that the length or distance that the drive shaft 18 (e.g., and/or the rotational device 20 secured thereto) is not limited by the sleeve 22 but rather the length of the path that the drive mechanism 23 is able to travel within/along the advancer 16. Theoretically, the advancer 16 could be made to have essentially any length. More practically, the advancer 16 and drive mechanism 23 may be sized/configured so that the throw length/distance of the drive shaft 18 (e.g., and/or the rotational device 20 secured thereto) may be on the order of about 1-50 cm, or about 5-30 cm, or about 8-20 cm, or about 12-18 cm, or about 15 cm or more, or more than about 8 cm, or more than about 10 cm.

Because both the drive shaft 18 and the sleeve 22 are attached to the drive mechanism 23, the axial position of the drive shaft 18 relative to the sleeve 22 is fixed. If the drive shaft 18 and the sleeve 22 are arranged so that a distal portion of the drive shaft 18 extends distally from the distal end of the sleeve 22 (e.g., as depicted in FIG. 1), the length of this distal portion will also remain fixed (e.g., the distal portion of the drive shaft 18 extending distally from the distal end of the sleeve 22 will have a fixed/constant length).

Also shown in FIG. 2 is that the drive shaft 18 may include a proximal or inner drive shaft portion 18a, a distal or drive coil portion 18b, and a joint or coupler 29. In some instances, a sheath or jacket (not shown) may be disposed along the outer surface of the proximal drive shaft portion 18a, the drive coil portion, or both. In instances where the drive shaft 18 includes the proximal drive shaft portion, the proximal drive shaft portion 18a may be attached to the drive mechanism 23.

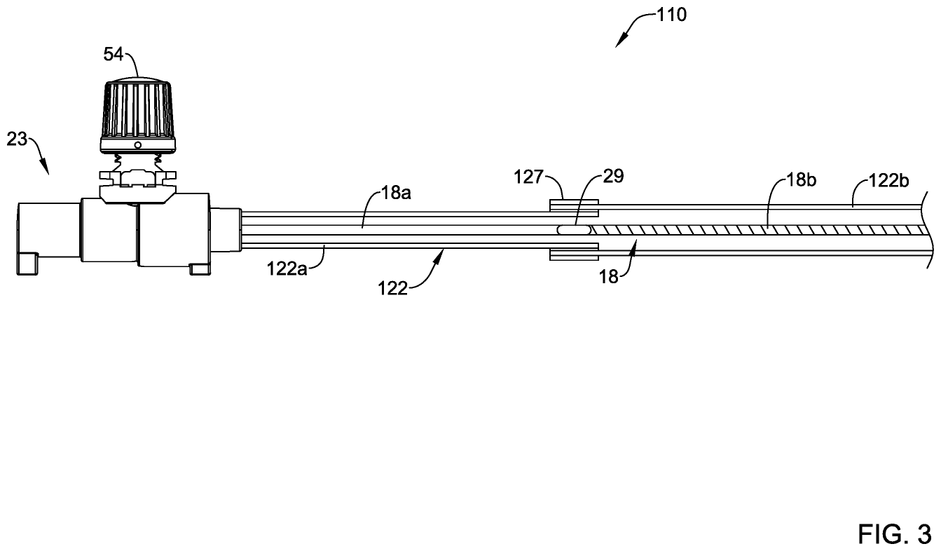

FIG. 3 is a partially cutaway view of a portion of an atherectomy system 110 that is similar in form and function to other systems disclosed herein. Here, the sleeve 122 may include a first or inner hypotube portion 122a, a second or outer hypotube portion 122b, and a connector and/or seal 127. This example merely demonstrates that alternative structures are contemplated for the sleeve 122 beyond just a single tubular struture.

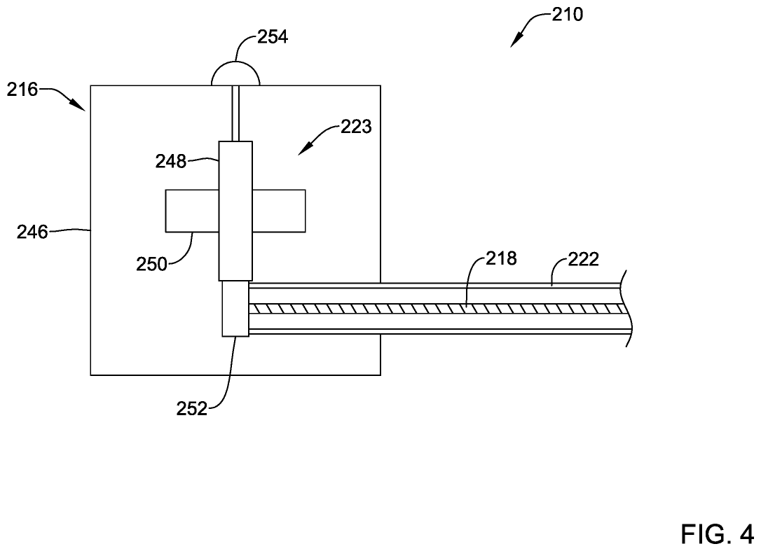

FIG. 4 is a partially cutaway view of a portion of an atherectomy system 210 that is similar in form and function to other systems disclosed herein. Here the advancer/handle 216 may include a housing 246. The drive mechanism 223 may include one or more arm/gear sections including a first section 248, a second section 250, and a third section 252 that may be movable relative to one another within the housing 246. In at least some instances, the section 248 may be slidable along or moveable relative to the section 250. This may include gears/teeth along sections 248 and/or section 250. Other configurations are contemplated including various rack structures, pinions, gear mechanisms, etc. The sheath 222 and the drive shaft 218 may be coupled to the drive mechanism 223 at section 252, which is coupled to the section 248. Thus, shifting/translating the section 248 shifts/translates the sheath 222 and the drive shaft 218. An actuator or button 254 may be coupled to one or more of the sections 248, 250, and 252 (e.g., the section 248).

The materials that can be used for the various components of the system 10 may include those commonly associated with medical devices. For example, the system may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device system, comprising:
an advancer including a drive mechanism;
a sleeve secured to the drive mechanism;
a drive shaft extending through the sleeve, the drive shaft having a proximal end region secured to the drive mechanism and a distal end region; and
a rotational device coupled to the distal end region of the drive shaft.

2. The medical device system of claim 1, wherein a distal section of the drive shaft extends distally from a distal end of the sleeve.

3. The medical device system of claim 2, wherein the distal section has a substantially constant length.

4. The medical device system of claim 1, wherein the advancer includes a housing and wherein the drive mechanism is configured to axially shift relative to the housing.

5. The medical device system of claim 4, wherein the drive mechanism includes a control member for axially shifting the drive mechanism.

6. The medical device system of claim 4, wherein the drive mechanism is configured to axially shift the drive shaft.

7. The medical device system of claim 4, wherein the drive mechanism is configured to axially shift the sleeve.

8. The medical device system of claim 1, wherein the drive shaft is axially fixed relative to the sleeve.

9. The medical device system of claim 1, wherein the drive shaft includes a coil portion.

10. The medical device system of claim 9, wherein the drive shaft includes a proximal portion coupled to the coil portion.

11. A rotational atherectomy system, comprising:
a catheter including a sleeve;
a coiled drive shaft extending through the sleeve, the coiled drive shaft having a proximal end region and a distal end region;
a rotational atherectomy device coupled to the distal end region of the coiled drive shaft;
a proximal drive shaft coupled to the proximal end region of the coiled drive shaft;
a drive mechanism coupled to the catheter;
wherein the proximal drive shaft is attached to the drive mechanism; and
wherein the sleeve is attached to the drive mechanism.

12. The rotational atherectomy system of claim 11, wherein a fixed-length section of the coiled drive shaft extends distally from a distal end of the sleeve.

13. The rotational atherectomy system of claim 11, wherein the coiled drive shaft is axially fixed relative to the sleeve.

14. The rotational atherectomy system of claim 11, wherein the proximal drive shaft is axially fixed relative to the sleeve.

15. The rotational atherectomy system of claim 11, wherein the drive mechanism includes a control member for axially shifting the coiled drive shaft.

16. A rotational atherectomy system, comprising:
a drive assembly including a drive mechanism and an actuator coupled to the drive mechanism;
a catheter coupled to the drive mechanism, the catheter including a sleeve;
a coiled drive shaft extending through the sleeve;
an atherectomy burr coupled to a distal end region of the coiled drive shaft;
wherein the sleeve is axially-fixed relative to the coiled drive shaft; and
wherein a fixed-length section of the coiled drive shaft extends distally from a distal end of the sleeve.

17. The rotational atherectomy system of claim 16, further comprising a proximal drive shaft coupled to a proximal end region of the coiled drive shaft.

18. The rotational atherectomy system of claim 17, wherein the proximal drive shaft is attached to the drive mechanism.

19. The rotational atherectomy system of claim 17, wherein the coiled drive shaft is axially fixed relative to the sleeve.

20. The rotational atherectomy system of claim 16, wherein the sleeve is attached to the drive mechanism.
